(19) United States
(12) Patent Application Publication
(10) Pub. No.: US 2019/0325767 A1
(43) Pub. Date: Oct. 24, 2019

(54) AN INTEGRATED SYSTEM AND INTERVENTION METHOD FOR ACTIVATING AND DEVELOPING WHOLE BRAIN COGNITION FUNCTIONS

(71) Applicant: COGNITIVES SKILLS PVT LTD., Bangalor (IN)

(72) Inventors: Nagaraja Nanjegowda Siddaramappa, Bangalore (IN); Meghna Manocha, Bangalore (IN)

(21) Appl. No.: 16/312,558

(22) PCT Filed: Jun. 20, 2017
(86) PCT No.: PCT/IN2017/050249

§ 371 Date: Dec. 21, 2018

(30) Foreign Application Priority Data
Jun. 21, 2016 (IN) 201641021234

(51) Int. Cl. G09B 5/06; A61B 5/053

(52) U.S. Cl. CPC G09B 5/06; A61B 5/4836; A61B 5/0533

(57) ABSTRACT

The present invention relates to an integrated system and a method to enhance the overall performance and efficiency of the brain by harmonizing and optimizing one or more functions. In one embodiment, this is accomplished by providing a first format frequency signal and a second format frequency signal in a diverse time interval to the participant to form a unison frequencies. Further, providing a third format frequency using the unison frequencies, where the resultant wave of frequency resonates with the internal neuronal transmitters and out of resonance the neurons start oscillating with the same frequency of the interfered signal.

### CROSS REFERENCE TO RELATED APPLICATION

The present application is the U.S. national phase of International Application No. PCT/IN2017/050249, filed Jun. 20, 2017, which is based on and claims priority to Indian Application No. 201641021234, filed Jun. 21, 2016, the disclosures of which are incorporated herein by reference in their entireties.

### FIELD OF THE INVENTION

The present invention relates to methods for enhancing learning skills and development of cognitive functions and skills in humans. More particularly, the invention relates to an integrated system and a method to enhance the overall performance and efficiency of the brain by harmonizing and optimizing one or more functions.

### BACKGROUND OF THE INVENTION

Cognition can be generally described as including at least three different components: attention, learning, and memory. Each of these components and their respective levels affect the overall level of a subject's cognitive ability. Cognitive functions depend on different regions in the brain to process and communicate information with each other. The information is typically in the form of electrical and chemical signals that are communicated along neural signaling pathways between these regions. The neural signaling pathways are composed of electrically active brain cells called neurons. In order to activate and take advantage of the same, there are several arts which teaches about the triggering of neurons in order to achieve various objectives. For example, prior art documents discloses about methods and apparatus for improving cognitive function within a human. The invention utilizes an implanted device i.e. electrodes into the brain tissues or stimulation of the scalp. Moreover, few documents teach about the utilization of electric stimulation, use cranial nerve stimulation for mood disorders and further use of sensory perceptual system for a language learning impaired individuals. All the above teaching discussed above are invasive and need some or the other physical component or installation in the human brain or body for achieving the objective. All the above teachings are more focussed on psychological illness people preferable for adults. Moreover, implementation of all the methodology and the devices which are used are expensive and need skilled person to operate the same.

It would, thus, be advantageous to provide a play and educational environment for children, including those children whose learning experiences have not been adequately developed, or for any medical reasons have been slow to develop. Such a virtual educational environment would be advantageous for several reasons: developmental milestones may be achieved more quickly; the child may learn fast and in efficient way and also it may be a seed for the development of imagination, consciousness, and communication in children.

### SUMMARY OF THE INVENTION

An aspect of the present invention is to address at least the above-mentioned problems and/or disadvantages and to provide at least the advantages described below.

Accordingly, in one aspect of the present invention provides a non-invasive method for improving cognition functions of a participant. The method including, providing a first format frequency signal in a first interval of time period to the participant, providing a second format frequency signal in a second interval of time period to the participant, the first format frequency and the second format frequency are unison frequencies, the first format frequency is lower frequency with respect to the second format frequency. Further, generating a third format frequency using the unison frequencies such that the third format frequency is the difference of the second format frequency and the first format frequency. The resultant wave of frequency resonates with the internal neuronal transmitters and out of resonance the neurons start oscillating with the same frequency of the interfered signal. Furthermore, providing a carrier signal as a contextual tone to the participant in order to carry the first format frequency and the second format frequency. The carrier signal has multiple frequencies.

In another aspect of the present invention relates to a computer-implemented auditory training exercise for improving cognition functions of a participant including observation and perception, intuition, memory, communication, decision making and problem solving which facilitate a holistic development of the participant. The method including, providing a first format frequency signal in a first interval of time period to the participant and a second format frequency signal in a second interval of time period to the participant. The first format frequency and the second format frequency are of low frequencies and are unison frequencies. Generating a third format frequency using the unison frequencies such that the third format frequency is the difference of the second format frequency and the first format frequency. Further, providing a carrier signal as a contextual tone to the participant in order to carrier the first format frequency and the second format frequency, the carrier signal has multiple frequencies such that frequencies are ordered in ascending order from low to high. The resultant wave of frequency resonates with the internal neuronal transmitters and out of resonance the neurons start oscillating with the same frequency of the interfered signal.

In another aspect of the present invention relates to a method for analysing activity performance of the brain of a participant. The method performs using a blindfold including a pair of eye shields to mask the eyes in order to prevent the vision of the participant. A plurality of low frequency tone disposed about the periphery of each of the ears of the participant at a frequency less than 20 Hz. A circuit means to drive a carrier signal along with the plurality of low frequencies, the carrier signal carry out as a contextual tone to the participant in order to carrier the plurality of frequencies ordered in ascending order from low to high. The resultant wave of frequency resonates with the internal neuronal transmitters and out of resonance the neurons start oscillating with the same frequency of the interfered signal.

In another aspect of the present invention relates to an auditory training exercise system for improving cognition functions of a participant and evaluating the effect of the same. The system including one or more computer and the computer configured to operating a computer processor to run a computer program including computer instructions processable by the computer processor, which when made available to the at least one computer, are operable to define a music based cognitive training utility that is operable to improve one or more cognitive functions of an individual. The music based cognitive training utility includes or defines a series of exercises consisting of musical exercises linked to improving cognitive function, and that cumulatively define a music based cognitive training module. The exercises includes, listening a plurality of frequency tones disposed about the periphery of each of the ears of the participant at a frequency less than 20 Hz, the plurality of frequency includes a carrier signal which act as a contextual tone to the participant and to carrier other plurality of frequencies, the resultant wave of frequencies resonates with the internal neuronal transmitters of the participant and out of resonance the neurons start oscillating with the same frequency of the interfered signal.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

The above and other aspects, features, and advantages of certain exemplary embodiments of the present invention will be more apparent from the following description taken in conjunction with the accompanying drawings in which:

FIG. 1 is a block diagram of a computer system for executing a program according to some embodiments of the present invention.

FIG. 2 is a block diagram of a computer network for executing a program according to some embodiments of the present invention.

FIG. 3 shows a flow chart of a non-invasive method for improving cognition functions of a participant, according to one embodiment of the present invention.

FIG. 4 shows a general system setup for generating a low frequency music or tone or harmony to the participant, according to exemplary embodiment of the present invention.

FIG. 5 shows a method as an example embodiment of the present invention for analysing activity performance of the brain of a participant.

FIG. 6 shows different modalities for measuring the physiological signals that reflects the effect that the intervention produces, according to one embodiment of the present invention.

FIG. 7 shows an encrypting and authenticating of the music delivery in a system environment, according to one embodiment of the present invention.

Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure.

Throughout the drawings, it should be noted that like reference numbers are used to depict the same or similar elements, features, and structures.

### DETAILED DESCRIPTION OF THE INVENTION

The following description with reference to the accompanying drawings is provided to assist in a comprehensive understanding of exemplary embodiments of the invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. In addition, descriptions of well-known functions and constructions are omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Figures discussed below, and the various embodiments used to describe the principles of the present disclosure in this patent document are by way of illustration only and should not be construed in any way that would limit the scope of the disclosure. Those skilled in the art will understand that the principles of the present disclosure may be implemented in any suitably arranged communications system. The terms used to describe various embodiments are exemplary. It should be understood that these are provided to merely aid the understanding of the description, and that their use and definitions in no way limit the scope of the invention. Terms first, second, and the like are used to differentiate between objects having the same terminology and are in no way intended to represent a chronological order, unless where explicitly stated otherwise. A set is defined as a non-empty set including at least one element.

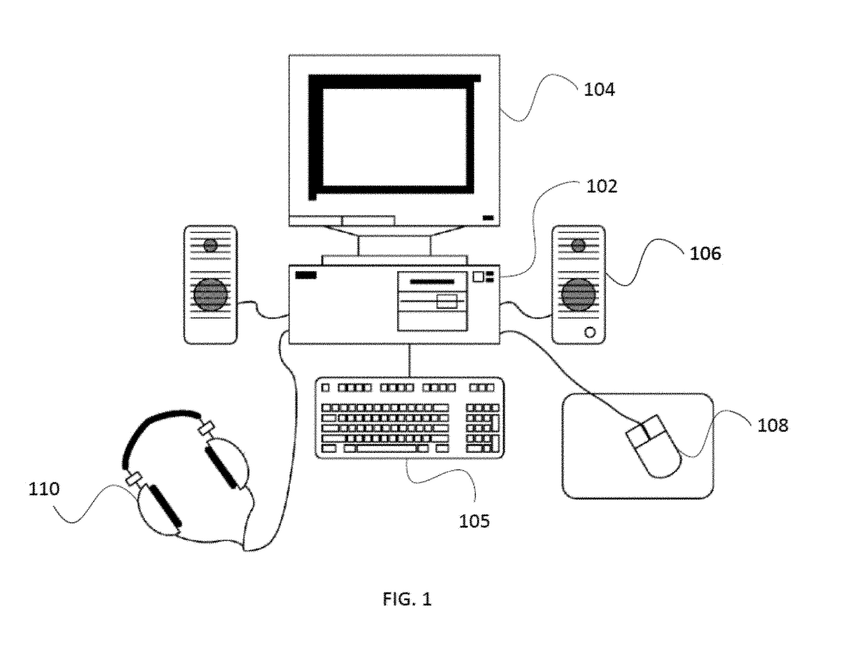

Referring to FIG. 1, a computer system 100 is shown for executing a computer program to train, or retrain an individual according to the present invention to enhance their memory and improve their cognition, where the term “cognition” refers to the speed, accuracy and reliability of processing of information, and attention and memory, and where the term “attention” refers to the facilitation of a target and/or suppression of a non-target over a given spatial extent, object-specific area or time window. The computer system 100 contains a computer 102, having a CPU, memory, hard disk and CD ROM drive (not shown), attached to a monitor 104. The monitor 104 provides visual prompting and feedback to the subject during execution of the computer program. Attached to the computer 102 are a keyboard 105, speakers 106, a mouse 108, and headphones 110. The speakers 106 and the headphones 110 provide auditory prompting and feedback to the subject during execution of the computer program. The mouse 108 allows the subject to navigate through the computer program, and to select particular responses after visual or auditory prompting by the computer program. The keyboard 105 allows an instructor to enter alpha numeric information about the subject into the computer 102. Although a number of different computer platforms are applicable to the present invention, embodiments of the present invention execute on either but not limited to any IBM compatible computers, or similarly configured computing devices such as set top boxes, PDA's, gaming consoles, etc.

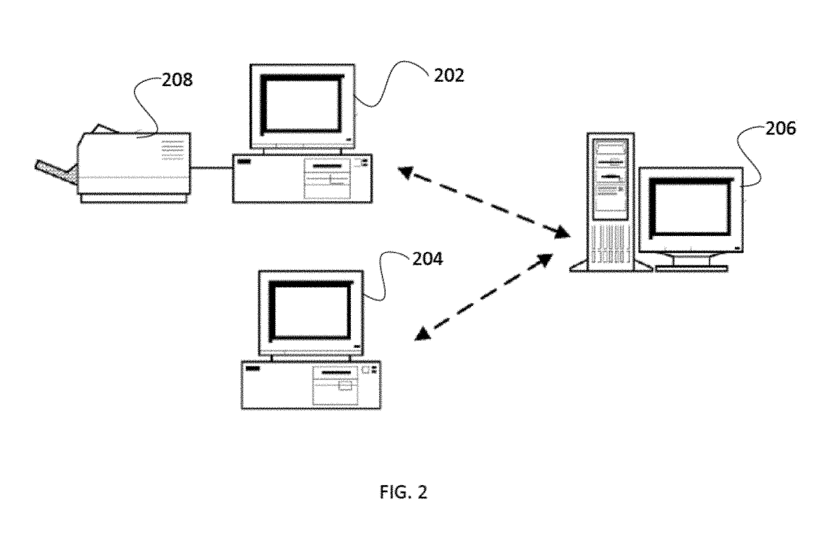

Now referring to FIG. 2, a computer network 200 is shown. The computer network 200 contains computers 202, 204, similar to that described above with reference to FIG. 1, connected to a server 206. The connection between the computers 202, 204 and the server 206 can be made via a local area network (LAN), a wide area network (WAN), or via modem connections, directly or through the Internet. A printer 208 is shown connected to the computer 202 to illustrate that a subject can print out reports associated with the computer program of the present invention. The computer network 200 allows information such as test scores, game statistics, and other subject information to flow from a subject's computer 202, 204 to a server 206. An administrator can then review the information and can then download configuration and control information pertaining to a particular subject, back to the subject's computer 202, 204.

Before providing a detailed description of the present invention, a brief overview of certain components of speech will be provided, along with an explanation of how these components are processed by subjects. Following the overview, general information on speech processing will be provided so that the reader will better appreciate the novel aspects of the present invention.

The invention relates to an integrated system, and intervention-based non-invasive program, that is aimed at enhancing the overall performance and efficiency of the brain by harmonizing and optimizing the various brain functions that results in the holistic development for the participant. In the current context, there is a one-sided thrust on academic performance both in the schools and homes. In the process, many brain functions are not exploited which have an important bearing on the holistic development of children. The proposed invention disclosure plays a complementary role for education/learning systems and it focuses on some of these cognitive functions such as observation and perception, memory, communication, decision making and problem solving that facilitate a holistic development of the participant.

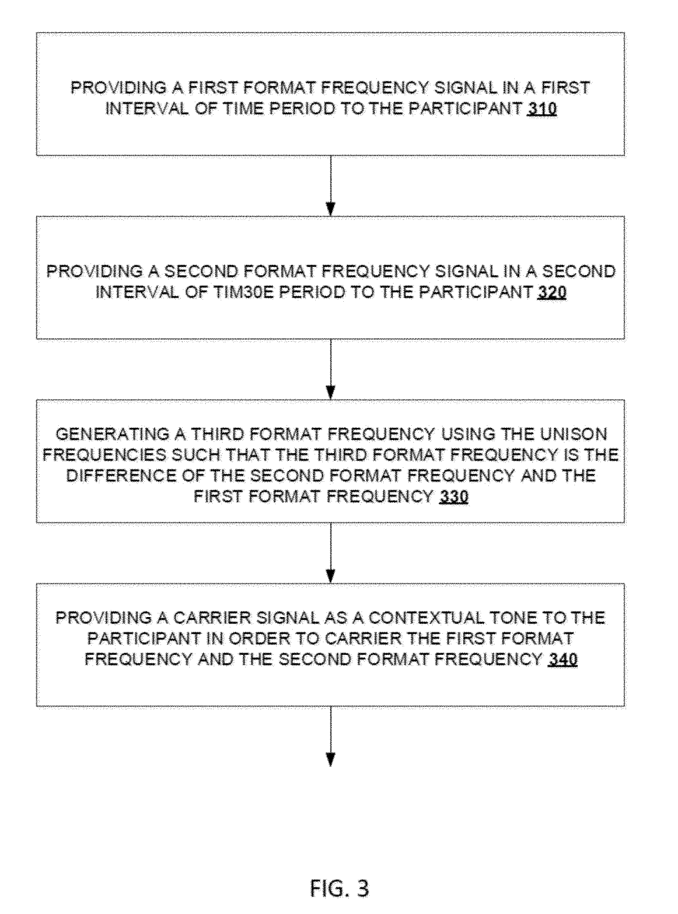

FIG. 3 shows a flow charts of a non-invasive method for improving cognition functions of a participant. At step 310, the method provides a first format frequency signal in a first interval of time period to the participant. At step 320, the method further provides a second format frequency signal in a second interval of time period to the participant. The first format frequency and the second format frequency are of low frequencies and are unison frequencies. The first format frequency is substantially lower frequency with respect to the second format frequency.

When sound is generated of one pure frequency ϑ₁ and another sound of pure frequency ϑ₂ such that ϑ₁≠ϑ₂ and ϑ₁<ϑ₂, these two pure tones when listened to in isolation is processed by the brain normally. In an example embodiment, human ear can process sound in the frequencies between 20 Hz to 20 KHz. Low frequencies below 20 Hz are known to affect the human brain bringing about increased learning capacity, intuition, memory, observation and other capacities such as intuitive skills. Since the frequencies below 20 Hz cannot be “heard” by the ear/auditory lobe (hearing system/cochleus), the invention provides an “auditory illusion”. As the two frequencies ϑ₁ and ϑ₂ are quite closer together, when listened to in unison, the resulting music undergoes a destructive interference in the brain and a low frequency of sound is generated in the auditory processing region of the brain giving it the illusion of listening to a third frequency such that the third frequency is ϑ=ϑ₂−ϑ₁. Since the two frequencies are quite close to each other, the resulting frequency ϑ is low frequency that is not audible to the human auditory system but is an auditory illusion created in the brain. Such a resulting wave of frequency ϑ resonates with the internal neuronal transmitters and out of resonance these neurons start oscillating with the same frequency ϑ of the interfered signal, as explained by step 330.

The frequency of the interfered signal is kept between 0.5 Hz to 32+Hz corresponding to the frequency bands of the neuronal states we wish to induce. For example, if the interfered signal is kept at lower than 4 Hz, the corresponding Delta state is produced by resonance in the listening participant's brain. Similarly, if the difference in frequencies between the two primary notes is kept between 4 Hz-7 Hz, the Alpha state is produced in the listener's neuronal system.

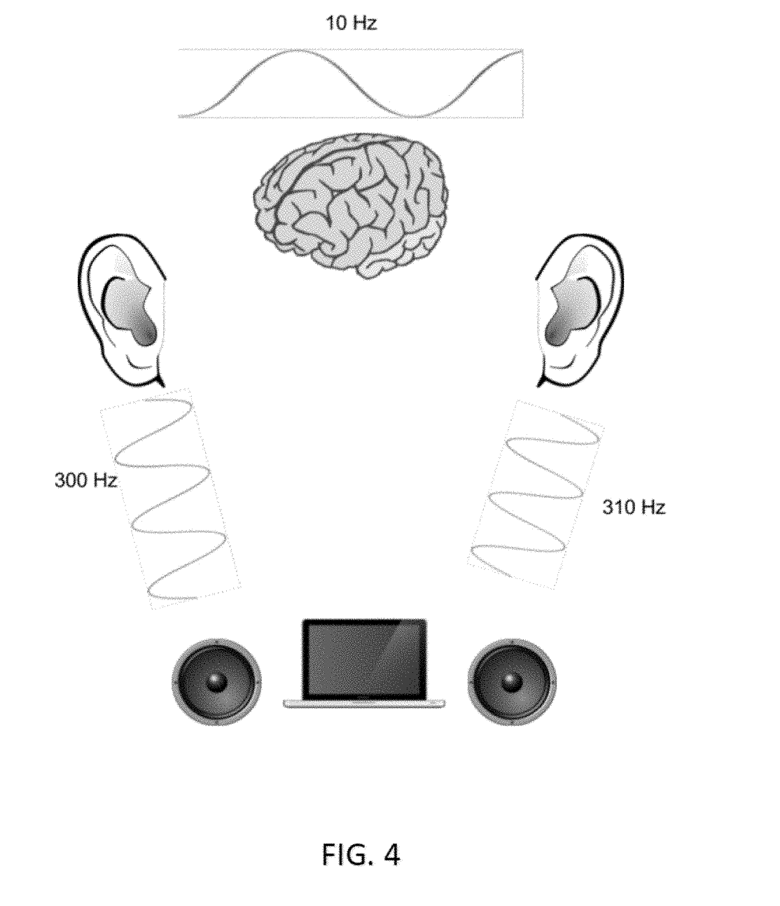

At step 340, the method provides a carrier signal as a contextual tone to the participant in order to carrier the first format frequency and the second format frequency. The carrier signal has multiple frequencies such that frequencies are ordered in ascending order from low to high. In this particular embodiments, the invention aims to provide the system, the right environment and training to the children to improve their brain functions related to observation, perception, attention and sensory augmentation. The uniqueness of this disclosure is to not use pure tone frequencies alone but also provide a carrier signal which is a background music to carrier these two pure tones. This carrier signal has multiple frequencies fi, i∈[1, N] such that the frequencies are ordered in ascending order from low to high with f₁<f_{N}. This carrier signal is modulated by the two pure frequencies ϑ₁ and ϑ₂ such that the interference of the three frequencies produces the extremely low frequency that is the desired state to be produced in the participant. The overall general system setup for generating the extra low frequency music is shown in FIG. 4.

In an example embodiment, a beat is generated when two sounds of slightly different frequencies interfere. The difference between the frequencies is the frequency of the beat. A beat is binaural, when two sounds (different frequencies and amplitude) is fed in two different ears through headphone or left-right speakers, and in brain, olivary body generates the beat (difference in frequency). A beat is monaural when two waveforms of same amplitude and slightly different frequencies are summed up. They occur in the open air and external to the ears. Beats which are manually created by turning ON & OFF evenly spaced tones. The invention method uses binaural beats (ELF) modulated with ‘Rag Desh’ to create a desired brainwave state for stimulating right brain. This stimulation results enhancement of cognitive skills relating to heightened sensory perception, improved memory capability, observation skills. It also helps creating a balanced state of mind and increases sensitivity.

In an example embodiment, the method employs ELF beats modulated with audible music to produce conducive brainwave patterns. This is primarily achieved by creating a positive environment and taking children into following states.

1. Alpha relaxation: In this session children listen to alpha frequency beats for about 30 minutes while they are comfortably seated with eyes closed. This stimulates intuition function.

2. Theta relaxation: In this session children listen to alpha-theta frequency beats for about 30 minutes. During this session, children are comfortably seated or lying down position with eyes closed. This stimulates intuition function at a deeper level. The learning space is darkened to reduce the distractions.

3. Beta relaxation: In this session children listen to beta modulated music for about 15 minutes and this is played while children are engaged in activities. This helps children to focus, concentration. This music supports creative function and keeps children energized. It is played in very low volume like a mild background music.

There are 5 brain waves in which the delta is the most minimal in the measure of frequency, which sounds like deeply penetrating drum beats, while highest is the Gamma wave which sounds like a high pitched flute. There are five types of brain waves with associated with different frequency ranges namely: Delta, (0.2/0.5 Hz to 3 to 4 Hz); Theta, (3 Hz to 7 Hz); Alpha, (7 or 8 Hz to 12 Hz); Beta (12 Hz to 30 Hz) and Gamma (30 Hz to 60 Hz/higher)

The lowest frequency which is almost 0.2 Hz or 0.5 Hz to around 3 to 4 Hz is called as Delta brainwave. In this state persons are not conscious of the external or physical awareness. It is a condition which is usually felt in the deepest state of meditation and can even be utilized for healing of mind. One can also say that it's a condition of dreamless or deep sleep state, where one often feels slow.

The frequency ranging from 3 Hz to 7 Hz is called Theta brainwave. These waves are usually dominant in deep meditation. It is a state where persons can access information beyond normal consciousness. It's an intuitive state where ideas and insights are created which is linked with learning or memories created in one's mind. It's a condition of mind where one can withdraw themselves from the consciousness of external world.

The frequency ranging from 7 Hz or 8 Hz to 12 Hz is called Alpha brainwave. It is a resting state of the mind where one can feel the condition of perfect calmness and integration of mind and body. It is a state where one's brain performs in the most optimized manner and utilizes its full potential. It is a state of mind where we have the maximum capacity to learn and gain knowledge.

The frequency ranging from 12 Hz to 30 Hz is called Beta waves. It is a condition in which the mind is wide awake and our attention is centered on externalities of the world surrounding us. It is a perspective where persons are actively involved in critical thinking and can feel different state of emotions and feelings like anxiety, excitement, etc. In this condition persons are capable of complex thought processes and can register new experience that gain in our lives. It's also a state where person spend a tremendous amount of energy.

The frequency ranging 30 Hz to 60 Hz or higher is called Gamma brainwave. It is a hyperactive state of mind where the brain can process information quite rapidly. It is a state where persons are depended on senses and creates perception of the external world from which one form ideas, create memories, form judgements and gain knowledge.

The invention and methodology that is proposed here is non-invasive and does not require any physical electrode implants or installations. It does not require any expensive device like magnetic or electric stimulator to activate the brain's cognitive functions. It can be administered by anyone who is trained and need not necessarily be a physician.

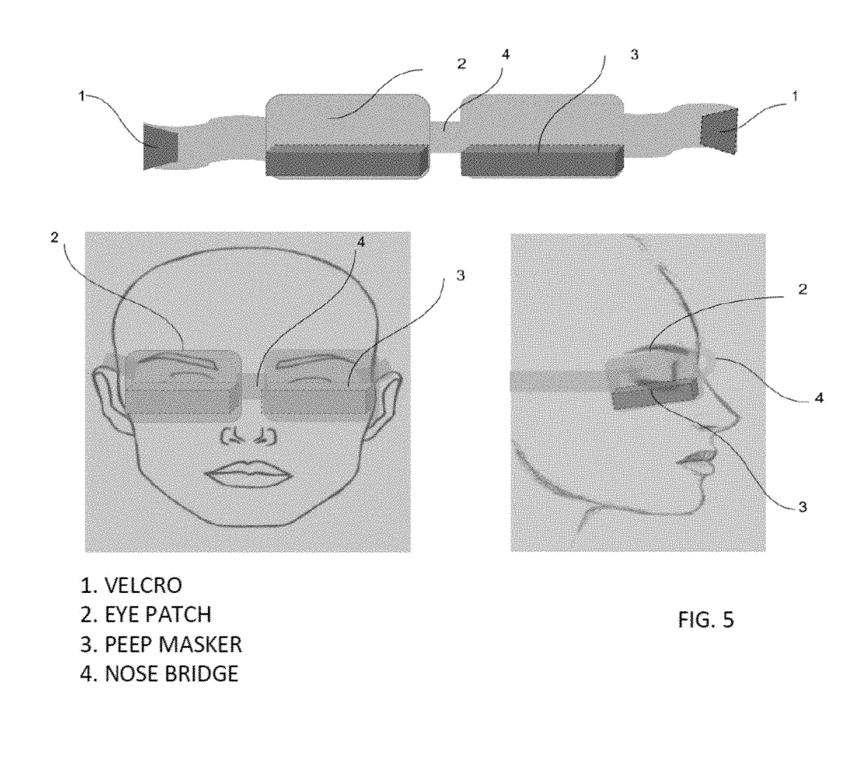

FIG. 5 shows an example embodiment of the present invention for analysing activity performance of the brain of a participant. The evaluation method for diagnosing activity performance using a blindfold which includes a pair of eye shields to mask the eyes in order to prevent the vision of the participant. The design in the embodiment involves an extra gear/contraption to provide support to the closed eyelids by not adding unnecessary pressure. In another exemplary embodiment, the blindfold has an added functionality of placing the blindfold in place by using velcros. Upon using the blindfold, the participant receives one or more of low frequency tone which is disposed about the periphery of each of the ears of the participant at a frequency less than 20 Hz. The method further includes a circuit means which drives a carrier signal along with the one or more of low frequencies. The carrier signal carry out as a contextual tone to the participant in order to carrier the plurality of frequencies ordered in ascending order from low to high. The resultant wave of frequency resonates with the internal neuronal transmitters and out of resonance the neurons start oscillating with the same frequency of the interfered signal.

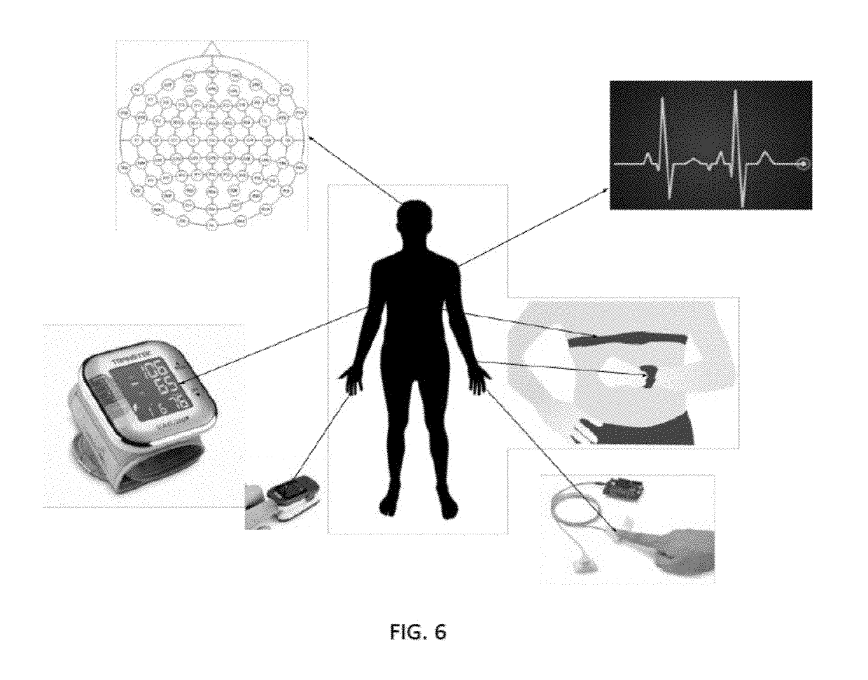

FIG. 6 shows different modalities for measuring the physiological signals that reflects the effect that the intervention produces, according to one embodiment of the present invention. The one or more physiological signal sensors are used for measuring the signals which reflects the effect of the procedure for improving cognition functions of a participant. One example of measurement might involve using the Galvanic Skin Response (GSR) Sensor to measure the Electrodermal Activity (EDA) of the participant that can give a quantitative measure of the sympathetic response of the participants and hence changes in the emotion of the participant. By using these sensors, one can obtain or measure the skill measurement using quantitative metrics, Intuition measurement and progress using observation Card, evaluating their reading and assimilation speed, evaluating their emotional resilience level, and can correlating heart-rate and brain-wave to identify when the participant takes decision before answering question.

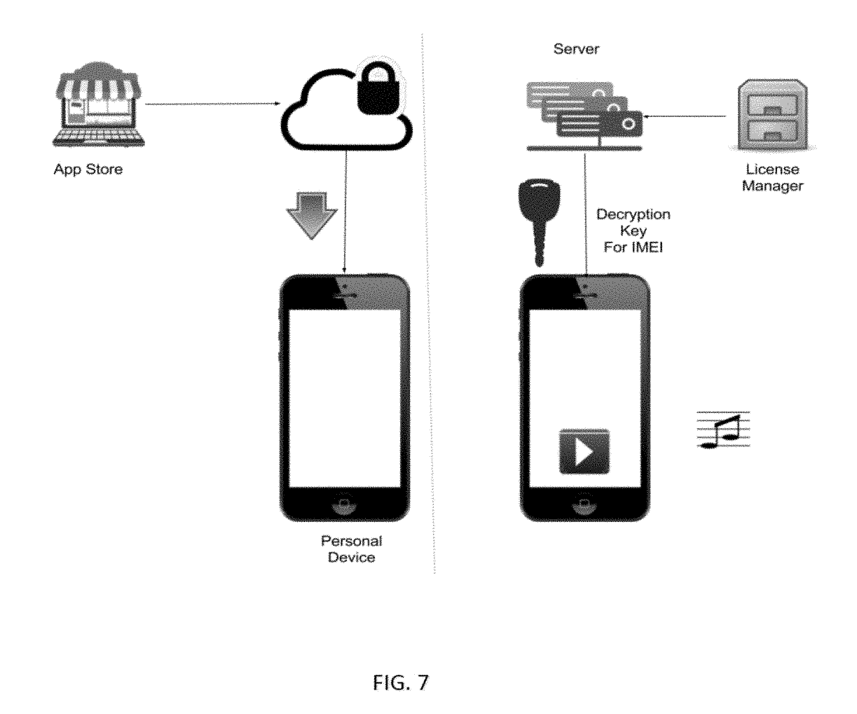

FIG. 7 shows an encrypting and authenticating of the music delivery in a system environment, according to one embodiment of the present invention. In the system broadly includes but not limited to client device or user device, a web server, key issuer, and right issuer. The user device, server, key issuer, right issuer, database all are connected in a network. A user's device may be or may include computer, mobile device, tablet or any other device that is capable of playing back music is installed with an application that generates the music. The user provides the device IMEI (International Mobile Equipment Identity) number for verification, the device connects to the internet and downloads an application to play the music from a storage device offline or online or from an online store. The downloaded and installed application when played by the user connects to the internet and connects to the license manager. The license manager verifies the device with the stored IMEI number to provide authorization for playing the music to the participant.

### Advantages

1. The proposed invention usually takes only 8 hours (50% reduction in time required) as compared to prior literature method where the first two days of entrainment takes almost 16 hours of training.

2. The proposed invention produces better results with music that is melodious and relaxing as compared to prior literature method uses quite loud music that can have side-effects like inducing headaches in the children.

3. The proposed invention is more relaxing sessions as compared to prior methods which are physically tiring for facilitators.

4. The proposed invention method has a tool to record the intuition test results as compared to prior methods does not have systematic tool to record the results.

Figures are merely representational and are not drawn to scale. Certain portions thereof may be exaggerated, while others may be minimized. Figures illustrate various embodiments of the invention that can be understood and appropriately carried out by those of ordinary skill in the art.

In the foregoing detailed description of embodiments of the invention, various features are grouped together in a single embodiment for the purpose of streamlining the disclosure. This method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus, the following claims are hereby incorporated into the detailed description of embodiments of the invention, with each claim standing on its own as a separate embodiment.

It is understood that the above description is intended to be illustrative, and not restrictive. It is intended to cover all alternatives, modifications and equivalents as may be included within the spirit and scope of the invention as defined in the appended claims. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein,” respectively.

1. A non-invasive method for improving cognition functions of a participant, the method comprising:
providing a first format frequency signal in a first interval of time period to the participant;
providing a second format frequency signal in a second interval of time period to the participant leading to an interference between first format frequency and the second format frequency, wherein the first format frequency is lower frequency with respect to the second format frequency,
the first format frequency and second format frequency being unison frequencies and the interference of the two frequencies generates an illusionary third format frequency in a participant brain which is the difference of the second format frequency and the first format frequency, the first format frequency and the second format frequency modulated over a carrier frequency;
wherein the third format frequency created in the brain resonates with the internal neuronal transmitters of the brain and due to resonance the neurons start oscillating with the same frequency of the third format frequency.

2. The method as claimed in claim 1,
wherein the carrier signal has multiple frequencies such that frequencies are ordered in ascending order from low to high.

3. The method as claimed in claim 1, wherein the first interval of time period is same as the second interval of time period.

4. The method as claimed in claim 1, wherein the generation of the third format frequency undergoes a destructive interference and provides an auditory illusion in the participant brain, having the third format frequency in the range of 0.5 Hz to 32+Hz.

5. The method as claimed in claim 1, wherein the difference of the first format frequency and the second format frequency is in range less than 20 Hz.

6. A The method as in claim 1, further comprising the steps of:
blindfolding with a pair of eye shields to mask the eyes in order to prevent the vision of the participant;
providing a plurality of tone disposed about the periphery of each of the ears of the participant resulting at a frequency of less than 20 Hz; and
providing a carrier signal through a circuit means, along with the plurality of low frequencies, the carrier signal carry out as a contextual tone to the participant in order to carrier the plurality of frequencies ordered in ascending order from low to high,
wherein the resultant wave of frequency resonates with the internal neuronal transmitters and due to resonance the neurons start oscillating with the same frequency of the interfered signal.

7. A computer-implemented auditory training exercise for improving cognition functions of a participant including observation and perception, memory, communication, decision making and problem solving which facilitate a holistic development of the participant, the method comprising:
providing a first format frequency signal in a first interval of time period to the participant;
providing a second format frequency signal in a second interval of time period to the participant leading to an interference between first format frequency and the second format frequency, wherein the first format frequency is lower frequency with respect to the second format frequency, the first format frequency and second format frequency being unison frequencies; wherein the interference of the two frequencies generates an illusionary third format frequency in a participant brain which is the difference of the second format frequency and the first format frequency, the first format frequency and the second format frequency modulated over a carrier frequency;
wherein the third format frequency created in the brain resonates with the internal neuronal transmitters of the brain and due to resonance the neurons start oscillating with the same frequency of the third format frequency.

8. An auditory training exercise system for improving cognition functions of a participant and evaluating the effect of the same, the system comprising:
at least one computer; and
the computer configured to operating a computer processor to run a computer program including computer instructions processable by the computer processor, which when made available to the at least one computer, are operable to define a music based cognitive training utility that is operable to improve one or more cognitive functions of an individual,
wherein the music based cognitive training utility includes or defines a series of exercises consisting of musical exercises linked to improving cognitive function, and that cumulatively define a music based cognitive training module, wherein exercises includes:
listening a plurality of frequency tones disposed about the periphery of each of the ears of the participant resulting in a frequency less than 20 Hz, the plurality of frequency includes a carrier signal which act as a contextual tone to the participant and to carrier other plurality of frequencies, the resultant wave of frequencies resonates with the internal neuronal transmitters of the participant and due to resonance the neurons start oscillating with the same frequency of the interfered signal.

9. The auditory training exercise system as claimed in claim 8, wherein the computer is operable to a plurality of physiological signal sensors in order to provide feedback to the system regarding the performance on various parameter including heart rate, reading and assimilation speed, emotional resilience level, brain wave etc.

10. The auditory training exercise system as claimed in claim 8, wherein the performance monitoring utility is operable to evaluate the individual based on recent learning.
